(19) United States
(12) Patent Application Publication
(10) Pub. No.: US 2026/0262955 A1
(43) Pub. Date: Sep. 10, 2026

(54) PHYSIOLOGICAL INFORMATION PROCESSING APPARATUS, PHYSIOLOGICAL INFORMATION PROCESSING METHOD, AND NON-TRANSITORY COMPUTER READABLE STORAGE MEDIUM

(71) Applicant: NIHON KOHDEN CORPORATION, Tokyo (JP)

(72) Inventor: Kou Adachi, Saitama (JP)

(73) Assignee: NIHON KOHDEN CORPORATION, Tokyo (JP)

(21) Appl. No.: 19/549,673

(22) Filed: Feb. 25, 2026

(30) Foreign Application Priority Data
Mar. 5, 2025 (JP) 2025-034357

(51) Int. Cl. A61B 5/024; A61B 5/00; A61B 5/1455

(52) U.S. Cl. CPC A61B 5/02405; A61B 5/02416; A61B 5/7264; A61B 5/7282; A61B 5/14551; A61B 5/741; A61B 5/742

(57) ABSTRACT

A physiological information processing apparatus includes an acquisition unit configured to acquire pulse wave data indicating a pulse wave waveform of a subject, and a detector configured to compare a first pulse wave waveform with a second pulse wave waveform next to the first pulse wave waveform, based on the pulse wave data, to detect an arrhythmia of the subject.

### CROSS-REFERENCE TO RELATED APPLICATIONS

This application is based on and claims priority under 35 USC 119 from Japanese Patent Application No. 2025-034357 filed on March 5, 2025, the entire content of which is incorporated herein by reference.

### TECHNICAL FIELD

The presently disclosed subject matter relates to a physiological information processing apparatus, a physiological information processing method, and a computer program.

### BACKGROUND ART

JP2022-148631A discloses a model that determines whether a particular type of arrhythmia occurs in a subject based on whether a predetermined type of peak is present in electrocardiogram waveform data acquired from the subject.

In recent years, the use of electrocardiogram transmitters has been reduced a hospital ward, and peripheral capillary oxygen saturation (SpO2) data of a subject may be acquired using a pulse oximeter instead of acquiring electrocardiogram data. However, in a measurement result of SpO2, the arrhythmia of the subject cannot be detected, and only a decrease in SpO2 as a result of the arrhythmia is detected.

### SUMMARY OF INVENTION

Aspect of non-limiting embodiments of the present disclosure relates to provide a physiological information processing apparatus, a physiological information processing method, and a computer program capable of detecting an arrhythmia of a subject without acquiring electrocardiogram data.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided a physiological information processing apparatus including:

an acquisition unit configured to acquire pulse wave data indicating a pulse wave waveform of a subject; and

a detector configured to compare a first pulse wave waveform with a second pulse wave waveform next to the first pulse wave waveform, based on the pulse wave data, to detect an arrhythmia of the subject.

### BRIEF DESCRIPTION OF DRAWINGS

Exemplary embodiment(s) of the present invention will be described in detail based on the following figures, wherein:

FIG. 1 is a block diagram illustrating a configuration of an information processing system according to an embodiment of the presently disclosed subject matter;

FIG. 2 is a flowchart illustrating an operation flow of a detector of a physiological information processing apparatus illustrated in FIG. 1;

FIG. 3 illustrates a method for detecting an arrhythmia by the detector of the physiological information processing apparatus illustrated in FIG. 1;

FIG. 4 illustrates a method for determining the presence or absence of atrial fibrillation by the detector illustrated in FIG. 1;

FIG. 5 illustrates a method for determining the presence or absence of premature contraction by the detector illustrated in FIG. 1; and

FIG. 6 illustrates a method for determining the presence or absence of tachycardia by the detector illustrated in FIG. 1.

### DESCRIPTION OF EMBODIMENTS

### Overall Configuration of Information Processing System

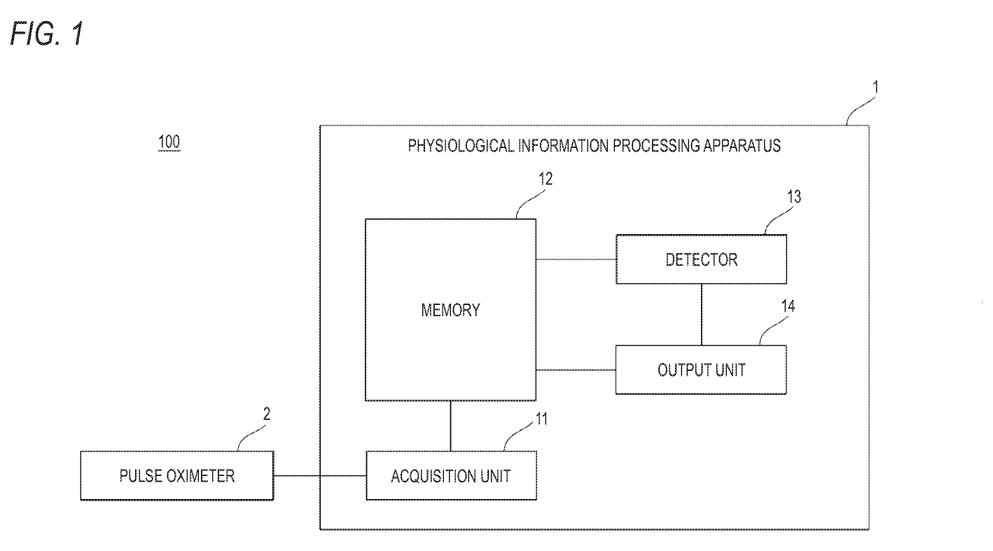

Hereinafter, an example of an embodiment of the presently disclosed subject matter will be described with reference to the drawings. FIG. 1 is a block diagram illustrating a configuration of an information processing system 100 according to an embodiment of the presently disclosed subject matter. The information processing system 100 can include a physiological information processing apparatus 1 and a pulse oximeter 2.

The physiological information processing apparatus 1 can include an acquisition unit 11, a memory 12, a detector 13, and an output unit 14. The pulse oximeter 2 is attached to a fingertip or the like of the subject. The pulse oximeter 2 is configured to measure a pulse wave of the subject and transmit pulse wave data to the physiological information processing apparatus 1.

The acquisition unit 11 in the physiological information processing apparatus 1 is configured to store the pulse wave data transmitted from the pulse oximeter 2 in the memory 12. The acquisition unit 11 may be an input interface. The detector 13 is configured to compare a pulse wave waveform with a pulse wave waveform next to the pulse wave waveform based on the pulse wave data stored in the memory 12 to detect an arrhythmia of the subject.

Based on the pulse wave data stored in the memory 12, the output unit 14 is configured to display, for example, a graph indicating a time-series change in peripheral capillary oxygen saturation (SpO2) of the subject on a monitor (not illustrated) or the like of the physiological information processing apparatus 1. For example, in a case where the arrhythmia of the subject is detected by the detector 13, the output unit 14 displays a detection result on a monitor or the like. The physiological information processing apparatus 1 may include a central processing unit (CPU), a read only memory (ROM), a random access memory (RAM), a hard disk drive (HDD), and the like. The CPU may function as the acquisition unit 11, the detector 13, and the output unit 14 and the like.

### Operation of Detector

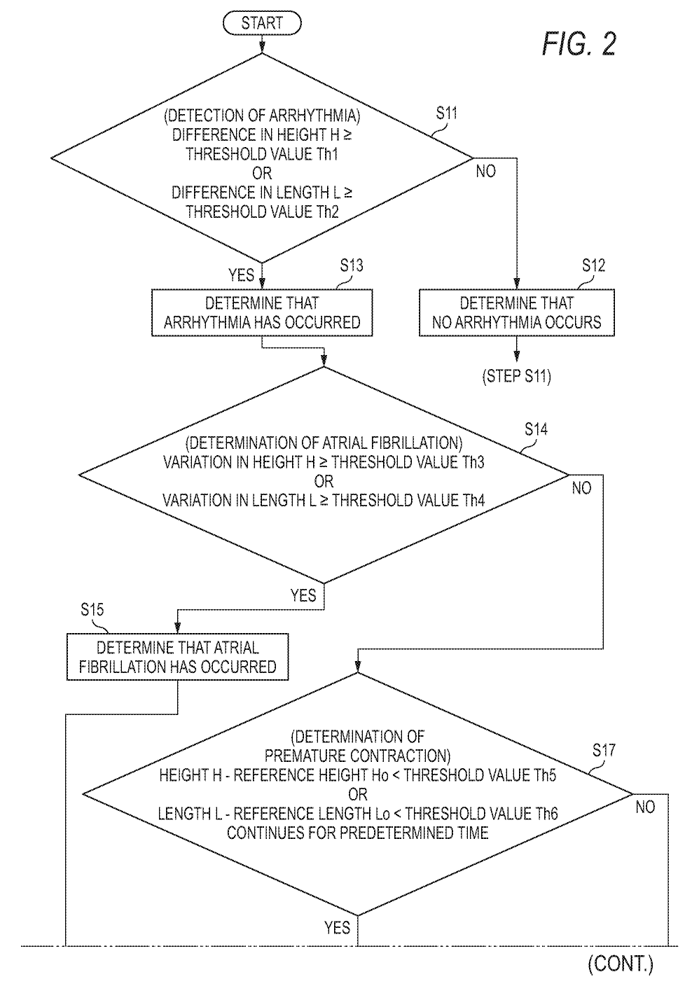

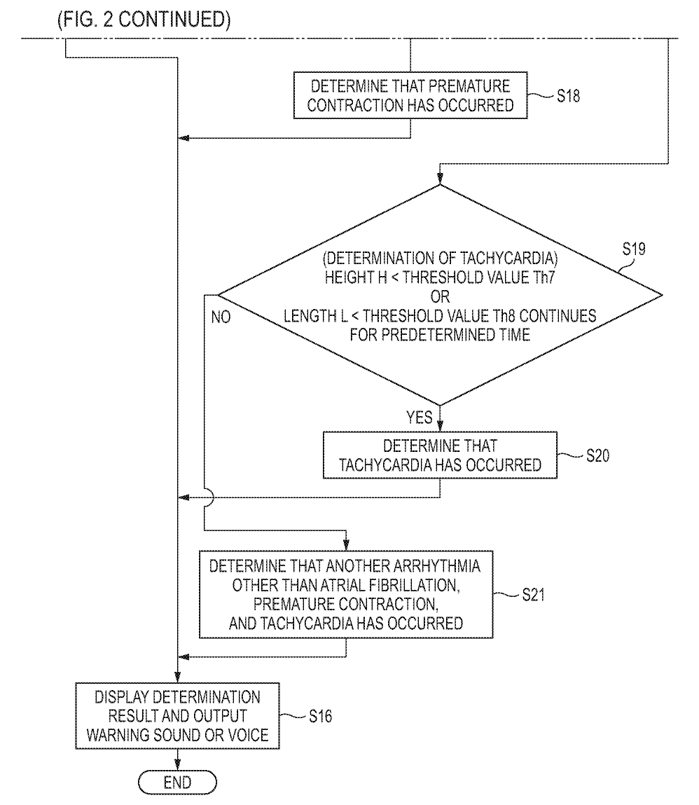

FIG. 2 is a flowchart illustrating an operation flow of the detector 13 of the physiological information processing apparatus 1 illustrated in FIG. 1. Hereinafter, the operation flow of the detector 13 will be described with reference to FIG. 2.

### Detection of Arrhythmia

First, the detector 13 detects the arrhythmia of the subject by periodically referring to the pulse wave data stored in the memory 12 and comparing the latest pulse wave waveform with the pulse wave waveform immediately before the latest pulse wave waveform, for example (step S11).

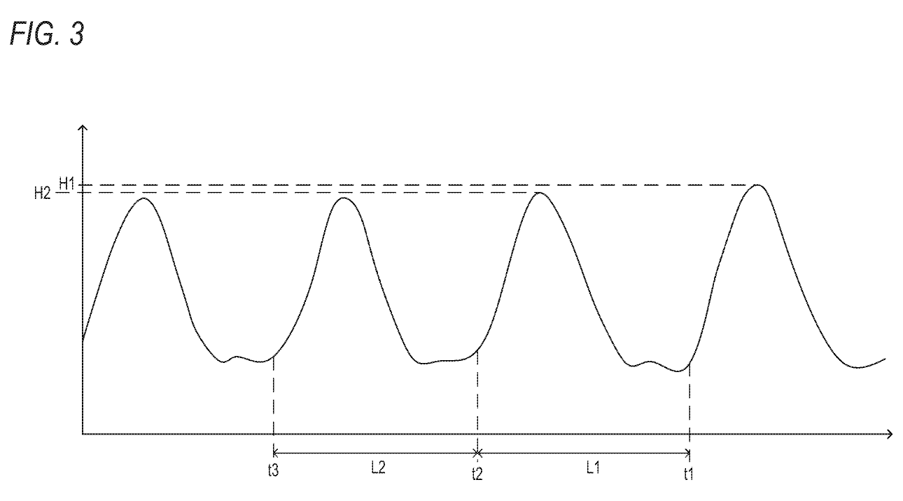

FIG. 3 illustrates a method for detecting the arrhythmia by the detector 13 of the physiological information processing apparatus 1 illustrated in FIG. 1. In a graph illustrated in FIG. 3, a horizontal axis represents time, and a vertical axis represents the height H of the pulse wave waveform. For example, the detector 13 is configured to specify the height H (a height H1 illustrated in FIG. 3) of the latest pulse wave waveform and the height H (a height H2 illustrated in FIG. 3) of the pulse wave waveform immediately before the latest pulse wave waveform.

In addition, the detector 13 is configured to specify a length L (a length L1 illustrated in FIG. 3) of an interval from a rising edge (a timing t2 illustrated in FIG. 3) of a pulse wave waveform immediately before the latest pulse wave waveform to a rising edge (a timing t1 illustrated in FIG. 3) of the latest pulse wave waveform. In addition, the detector 13 is configured to specify a length L (a length L2 illustrated in FIG. 3) of an interval from a rising edge (a timing t3 illustrated in FIG. 3) of a pulse wave waveform two pulse wave waveforms before the latest pulse wave waveform to the rising edge (the timing t2 illustrated in FIG. 3) of a pulse wave waveform immediately before the latest pulse wave waveform.

The detector 13 is configured to determine whether a difference between the specified heights H, that is, a difference between the height H1 and the height H2 illustrated in FIG. 3 is equal to or greater than a threshold value (a first threshold value) Th1. In addition, the detector 13 is configured to determine whether a difference between the specified lengths L, that is, a difference between the length L1 and the length L2 illustrated in FIG. 3 is equal to or greater than a threshold value (a second threshold value) Th2.

Then, the detector 13 determines that the difference in height H is less than the threshold value Th1 and the difference in length L of the interval is less than the threshold value Th2 ("NO" in step S11). In this case, the detector 13 determines that no arrhythmia occurs in the subject (step S12), and periodically repeats, for example, operations from step S11 onward.

In the example illustrated in FIG. 3, there is no large change in the height H and the length L of the interval in a plurality of continuous pulse wave waveforms. In such a case, the detector 13 determines that the difference in height H is less than the threshold value Th1 and the difference in length L of the interval is less than the threshold value Th2, and determines that no arrhythmia occurs in the subject.

On the other hand, in step S11, it is assumed that at least one of the condition that the difference in height H is equal to or greater than the threshold value Th1 and the condition that the difference in length L of the interval is equal to or greater than the threshold value Th2 is satisfied ("YES" in step S11). In this case, the detector 13 determines that the arrhythmia has occurred in the subject (step S13).

The detector 13 may be configured to detect the arrhythmia using both the difference in height H and the difference in length L of the interval. That is, the detector 13 may be configured to determine that the arrhythmia has occurred in the subject when the difference in height H is equal to or greater than the threshold value Th1 and the difference in length L of the interval is equal to or greater than the threshold value Th2.

As described above, the detector 13 can detect the arrhythmia of the subject without acquiring the electrocardiogram data by comparing the pulse wave waveform of the subject with the pulse wave waveform next to the pulse wave waveform. The arrhythmia of the subject can be detected not only when the subject is in the hospital ward but also when the subject is at home.

The pulse wave data used for detecting an arrhythmia in the physiological information processing apparatus 1 is not limited to the pulse wave data used for measuring SpO2. However, in the pulse wave data used for measuring SpO2, when the arrhythmia has occurred in the subject, a change is likely to occur between continuous pulse wave waveforms. Therefore, the acquisition unit 11 is preferably configured to acquire the pulse wave data used for measuring SpO2. In addition, the arrhythmia of the subject can be detected using a pulse oximeter that is often used in a hospital ward or the like without providing a new measuring instrument for detecting the arrhythmia.

### Determination of Type of Arrhythmia (1) Determination of Presence or Absence of Atrial Fibrillation

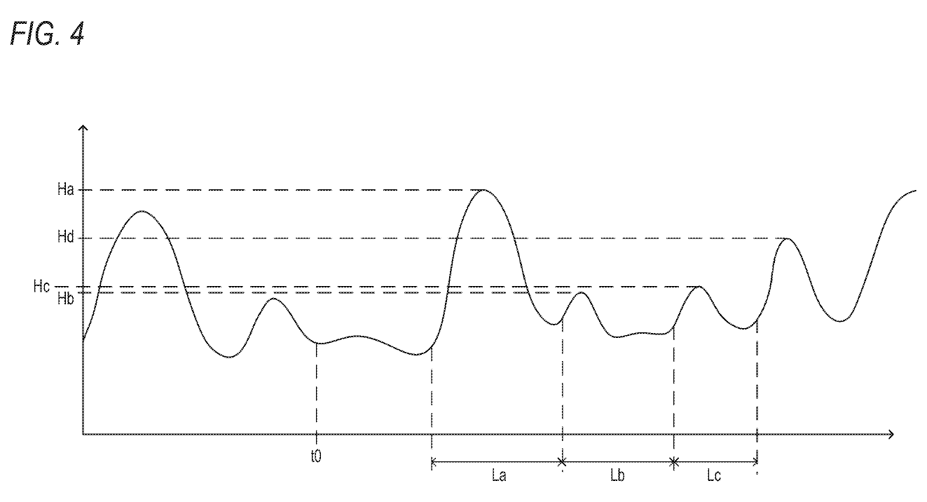

Next, after determining that the arrhythmia has occurred (step S13), the detector 13 determines the type of the detected arrhythmia. FIG. 4 illustrates a method for determining the presence or absence of atrial fibrillation by the detector 13 illustrated in FIG. 1. In a graph illustrated in FIG. 4, a horizontal axis represents time, and a vertical axis represents the height H of the pulse wave waveform.

Specifically, when the arrhythmia of the subject is detected, the detector 13 determines whether the atrial fibrillation has occurred in the subject (step S14). More specifically, the detector 13 is configured to refer to the pulse wave data stored in the memory 12 and specify the height H of the pulse wave waveform and the length L of the interval between the preceding and following pulse wave waveforms for each of the plurality of pulse wave waveforms in a predetermined time after a timing (a timing t0 illustrated in FIG. 4) at which the arrhythmia is detected.

The detector 13 determines whether a variation in the height H of the pulse wave waveform is equal to or greater than a threshold value Th3 and whether a variation in the length L of the interval between the preceding and following pulse wave waveforms is equal to or greater than a threshold value Th4.

Then, it is assumed that at least one of the condition that the variation in the height H of the pulse wave waveform is equal to or greater than the threshold value Th3 and the condition that the variation in the length L of the interval is equal to or greater than the threshold value Th4 is satisfied ("YES" in step S14). In this case, the detector 13 determines that the atrial fibrillation has occurred in the subject (step S15).

For example, as illustrated in FIG. 4, it is assumed that the variation in the height H of the pulse wave waveform and the length L of the interval is large after the timing t0 at which the arrhythmia is detected. In such a case, the detector 13 determines that the variation in the height H of the pulse wave waveform is equal to or greater than the threshold value Th3. The detector 13 determines that the variation in the length L of the interval is equal to or greater than the threshold value Th4. Then, the detector 13 determines that the atrial fibrillation has occurred in the subject.

The detector 13 may be configured to determine whether the atrial fibrillation has occurred in the subject using both the height H of the pulse wave waveform and the length L of the interval.

That is, the detector 13 may be configured to determine that the atrial fibrillation has occurred in the subject in a case where the variation in the height H of the pulse wave waveform is equal to or greater than the threshold value Th3 and the variation in the length L of the interval is equal to or greater than the threshold value Th4 in the predetermined time after the arrhythmia is detected. With such a configuration, it is possible to improve detection accuracy of the atrial fibrillation. In addition, in such a configuration, the length of the predetermined time required for determining the presence or absence of the atrial fibrillation may be shortened.

Instead of calculating the variation in the height H of the pulse wave waveform, the detector 13 may be configured to calculate the variation in the difference in the height H of the pulse wave waveform. Instead of calculating the variation in the length L of the interval between the preceding and following pulse wave waveforms, the detector 13 may be configured to calculate the variation in the difference between the lengths L of the intervals between the preceding and following pulse wave waveforms.

Specifically, as illustrated in FIG. 4, the height H of the pulse wave waveform of a first beat after the arrhythmia is detected is "Ha", the height H of the pulse wave waveform of a second beat is "Hb", the height H of the pulse wave waveform of a third beat is "Hc", and the height H of the pulse wave waveform of a fourth beat is "Hd". In this case, the detector 13 can calculate, for example, a difference between the height Ha and the height Hb, a difference between the height Hb and the height Hc, a difference between the height Hc and the height Hd, ....

The length L of the interval between the pulse wave waveform of the first beat and the pulse wave waveform of the second beat after the arrhythmia is detected is "La", the length L of the interval between the pulse wave waveform of the second beat and the pulse wave waveform of the third beat is "Lb", and the length L of the interval between the pulse wave waveform of the third beat and the pulse wave waveform of the fourth beat is "Lc". In this case, the detector 13 can calculate, for example, a difference between the length La and the length Lb, a difference between the length Lb and the length Lc, a difference between the length Lc and the length Ld, ....

Then, in this case, for example, the detector 13 determines that the atrial fibrillation has occurred in the subject when at least one of a condition that the variation in the difference in the height H of the pulse wave waveform is equal to or greater than a threshold value and a condition that the variation in the difference in the length L of the interval is equal to or greater than a threshold value is satisfied.

Referring again to FIG. 2, as the detector 13 determines the presence or absence of atrial fibrillation, the detector 13 outputs the determination result to the output unit 14. When receiving the determination result by the detector 13, the output unit 14 displays information indicating the determination result on a monitor or the like, or outputs a warning sound, a voice, or the like (step S16).

### (2) Determination of Presence or Absence of Premature Contraction

In step S14, it is assumed that at least one of the condition that the variation in the height H of the pulse wave waveform is less than the threshold value Th3 and the condition that the variation in the length L of the interval is less than the threshold value Th4 is satisfied ("NO" in step S14). In this case, the detector 13 determines, for example, whether the premature contraction has occurred in the subject (step S17).

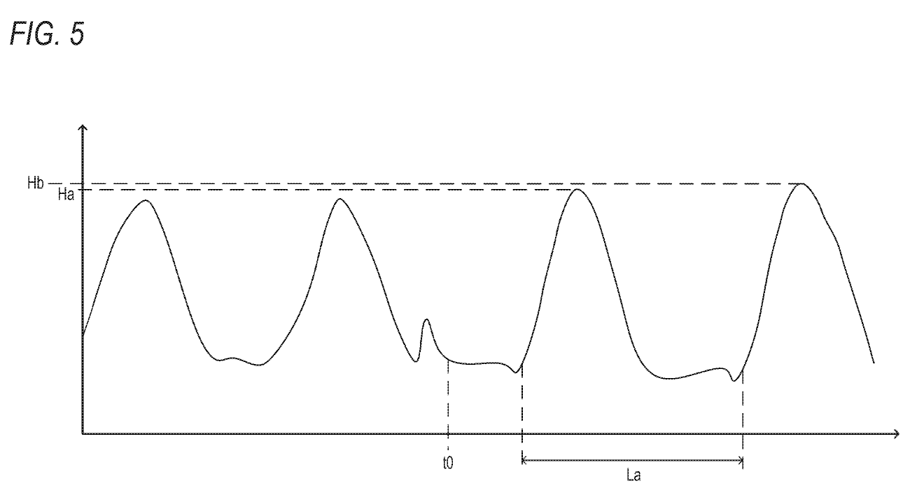

FIG. 5 illustrates a method for determining the presence or absence of the premature contraction by the detector 13 illustrated in FIG. 1. In a graph illustrated in FIG. 5, a horizontal axis represents time, and a vertical axis represents the height H of the pulse wave waveform.

Here, the memory 12 illustrated in FIG. 1 stores reference data indicating the height of the pulse wave waveform of the subject (hereinafter, referred to as a "reference height Ho") and the length of the interval between the preceding and following pulse wave waveforms (hereinafter, referred to as a "reference length Lo") in a state in which no arrhythmia occurs in the subject (hereinafter, referred to as a "normal state"). The reference height Ho is an average value or a median value of the heights H1, H2, ... of the pulse wave waveforms in the normal state as illustrated in FIG. 3. The reference length Lo is an average value or a median value of the lengths L1, L2, ... of the intervals in the normal state as illustrated in FIG. 3.

The detector 13 is configured to refer to the pulse wave data and the reference data stored in the memory 12, and determine whether a state in which the difference between the height H of the pulse wave waveform and the reference height Ho is less than a threshold value (a third threshold value) Th5 continues for a predetermined time after the timing t0 at which the arrhythmia is detected. In addition, the detector 13 is configured to determine whether a state where the difference between the length L of the interval and the reference length Lo is less than a threshold value (a fourth threshold value) Th6 continues for the predetermined time.

Then, it is assumed that at least one of the state in which the difference between the height H of the pulse wave waveform and the reference height Ho is less than the threshold value Th5 continues and the state where the difference between the length L of the interval and the reference length Lo is less than the threshold value Th6 continues is satisfied ("YES" in step S17). In this case, the detector 13 determines that the premature contraction has occurred in the subject (step S18).

For example, as illustrated in FIG. 5, it is assumed that the same pulse wave waveform as the pulse wave waveform in the normal state continues for the predetermined time after the timing t0 at which the arrhythmia is detected. That is, it is assumed that the heights Ha, Hb, ... of the pulse wave waveforms after the arrhythmia is detected are values close to the reference height Ho, and the lengths La, ... of the intervals after the arrhythmia is detected are values close to the reference length Lo.

In such a case, the detector 13 determines that the state in which the difference between the height H of the pulse wave waveform and the reference height Ho is less than the threshold value Th5 has continued for the predetermined time. In addition, the detector 13 determines that the state in which the difference between the length L of the interval and the reference length Lo is less than the threshold value Th6 continues for the predetermined time. Then, the detector 13 determines that the premature contraction has occurred in the subject.

Note that the detector 13 may be configured to determine that the state in which the difference between the height H of the pulse wave waveform and the reference height Ho is less than the threshold value Th5 has continued for the predetermined time in a case where the difference is equal to or greater than the threshold value Th5 for only a short period of time during the predetermined time. For example, it is assumed that the difference is less than the threshold value Th5 in 90% or more of the predetermined time, and the difference is equal to or greater than the threshold value Th5 in less than 10% of the predetermined time. In this case, the detector 13 can determine that the state in which the difference is less than the threshold value Th5 has continued for the predetermined time.

The detector 13 may determine that the state in which the difference between the length L of the interval and the reference length Lo is less than the threshold value Th6 has continued for the predetermined time in a case where the difference is equal to or greater than the threshold value Th6 for only a short period of time during the predetermined time. For example, it is assumed that the difference is less than the threshold value Th6 in 90% or more of the predetermined time, and the difference is equal to or greater than the threshold value Th6 in less than 10% of the predetermined time. In this case, the detector 13 can determine that the state in which the difference is less than the threshold value Th6 has continued for the predetermined time.

Referring again to FIG. 2, the detector 13 is configured to output the determination result to the output unit 14. Then, the operation of step S16 is performed by the output unit 14.

The detector 13 may be configured to determine whether the premature contraction has occurred in the subject by using both the difference between the height H of the pulse wave waveform and the reference height Ho and the difference between the length L of the interval and the reference length Lo.

That is, the detector 13 may be configured to determine that the atrial fibrillation has occurred in the subject when a state in which the difference between the height H of the pulse wave waveform and the reference height Ho is less than the threshold value Th5 and the difference between the length L of the interval and the reference length Lo is less than the threshold value Th6 continues for the predetermined time after the arrhythmia is detected. In such a configuration, it is possible to improve detection accuracy of the premature contraction. In addition, in such a configuration, the length of the predetermined time required for determining the presence or absence of the premature contraction may be shortened.

### (3) Detection of Tachycardia

In step S17, it is assumed that at least one of the condition that the difference between the height H of the pulse wave waveform and the reference height Ho is equal to or greater than the threshold value Th5 and the condition that the difference between the length L of the interval and the reference length Lo is equal to or greater than the threshold value Th6 is satisfied ("NO" in step S17). In this case, for example, the detector 13 determines whether the tachycardia occurs in the subject (step S19).

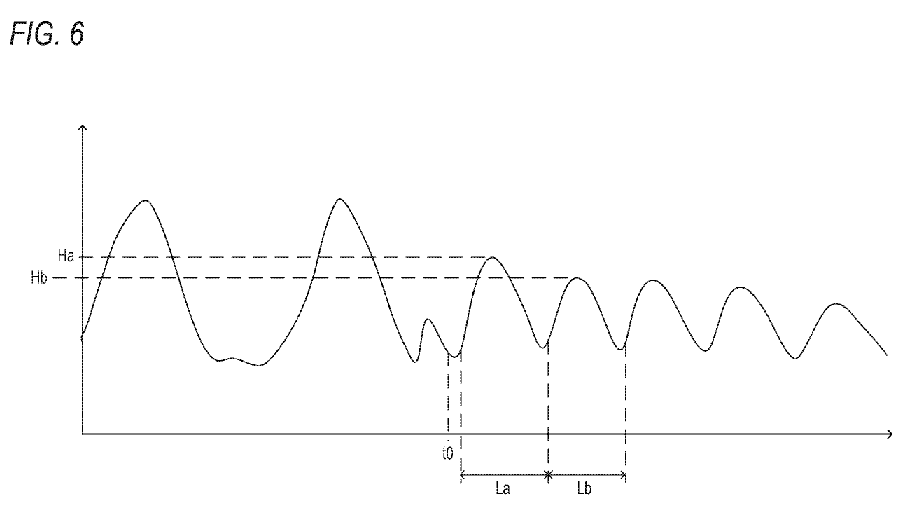

FIG. 6 illustrates a method for determining the presence or absence of the tachycardia by the detector 13 illustrated in FIG. 1. In a graph illustrated in FIG. 6, a horizontal axis represents time, and a vertical axis represents the height H of the pulse wave waveform.

More specifically, for example, the detector 13 is configured to refer to the pulse wave data stored in the memory 12, and determine whether a state in which the height H of the pulse wave waveform is less than the threshold value (a fifth threshold value) Th7 continues for a predetermined time after the timing t0 at which the arrhythmia is detected. In addition, the detector 13 is configured to determine whether a state in which the length L of the interval is less than a threshold value (a sixth threshold value) Th8 continues for the predetermined time.

Then, it is assumed that at least one of the state in which the height H of the pulse wave waveform is less than the threshold value Th7 continues and the state in which the length L of the interval is less than the threshold value Th8 continues is satisfied ("YES" in step S19). In this case, the detector 13 determines that the tachycardia has occurred in the subject (step S20).

For example, as illustrated in FIG. 6, it is assumed that a state in which the height H of the pulse wave waveform is low and the length L of the interval is short continues for the predetermined time after the timing t0 at which the arrhythmia is detected. That is, it is assumed that the heights Ha, Hb, ... of the pulse wave waveforms after the arrhythmia is detected are low, and the lengths La, Lb, ... of the intervals after the arrhythmia is detected are short.

In such a case, the detector 13 determines that the state in which the height H of the pulse wave waveform is less than the threshold value Th7 has continued for the predetermined time. In addition, the detector 13 determines that the state in which the length L of the interval is less than the threshold value Th8 has continued for the predetermined time. Then, the detector 13 determines that the tachycardia has occurred in the subject.

Referring again to FIG. 2, the detector 13 is configured to output the determination result to the output unit 14. Then, the operation of step S16 is performed by the output unit 14.

On the other hand, it is assumed that at least one of the condition that the height H of the pulse wave waveform is equal to or greater than the threshold value Th7 and the condition that the length L of the interval is equal to or greater than the threshold value Th8 is satisfied ("NO" in step S19). In this case, the detector 13 determines that another arrhythmia other than the atrial fibrillation, the premature contraction, and the tachycardia has occurred in the subject (step S21). Then, the detector 13 outputs the determination result to the output unit 14, and the operation of step S16 is performed by the output unit 14.

The detector 13 may be configured to determine whether the tachycardia has occurred in the subject using both the height H of the pulse wave waveform and the length L of the interval between the preceding and following pulse wave waveforms.

That is, the detector 13 may be configured to determine that the tachycardia has occurred in the subject when a state in which the height H of the pulse wave waveform is less than the threshold value Th7 and the length L of the interval between the preceding and following pulse wave waveforms is less than the threshold value Th8 continues for the predetermined time after the arrhythmia is detected. With such a configuration, it is possible to improve detection accuracy of the tachycardia. In addition, in such a configuration, the length of the predetermined time required for determining the presence or absence of the tachycardia may be shortened.

In the above-described example, the detector 13 performs determination processes in the order of the determination of the atrial fibrillation (step S14), the determination of the premature contraction (step S17), and the determination of the tachycardia (step S19). However, the determination processes by the detector 13 are not limited to such an order. Further, these determination processes may be performed in parallel.

Although the embodiment of the presently disclosed subject matter has been described above, it is needless to say that the technical scope of the presently disclosed subject matter should not be construed as being limited to the description of the embodiment. The present embodiment is merely an example, and it is understood by those skilled in the art that various modifications of the embodiment are possible within the scope of the disclosed subject matter described in the claims. The technical scope of the presently disclosed subject matter should be determined based on the scope of the disclosed subject matter described in the claims and equivalents thereof.

The processes of the physiological information processing apparatus 1 according to the embodiment can be implemented as a computer program that operates in the physiological information processing apparatus 1. That is, the physiological information processing apparatus 1 can include a processor such as a CPU.

The program is stored in a non-transitory computer-readable medium and can be read by a computer. Examples of the non-transitory computer-readable medium include a magnetic recording medium, a magneto-optical recording medium, a CD-ROM, a CD-R, a CD-R/W, and a semi-conductor memory (including an EPROM and a flash ROM). The program may be read by a computer using various types of temporary computer-readable media. Examples of the temporary computer-readable medium include an electric signal, an optical signal, and an electromagnetic wave. The temporary computer-readable medium can supply a program to the computer via a wired communication path such as an electric wire and an optical fiber or a wireless communication path.

As described above, the present specification discloses the following matters.

(1) A physiological information processing apparatus including:

an acquisition unit configured to acquire pulse wave data indicating a pulse wave waveform of a subject; and

a detector configured to compare a first pulse wave waveform with a second pulse wave waveform next to the first pulse wave waveform, based on the pulse wave data, to detect an arrhythmia of the subject.

With such a configuration, it is possible to detect the arrhythmia of the subject using the pulse wave data of the subject without acquiring electrocardiogram data.

(2) The physiological information processing apparatus according to (1), in which the detector is configured to detect the arrhythmia of the subject, based on at least one of a height of the pulse wave waveform and a length of an interval between the first pulse wave waveform and the second pulse wave waveform.

With such a configuration, the arrhythmia of the subject can be detected without performing a complicated process. In addition, when the arrhythmia is detected using both the height of the pulse wave waveform and the interval between the continuous pulse wave waveforms, detection accuracy can be improved.

(3) The physiological information processing apparatus according to (2), in which after the arrhythmia of the subject is detected, the detector is configured to determine whether atrial fibrillation has occurred in the subject, based on at least one of a variation in height of each pulse wave waveform in a predetermined time, a variation in difference in height between preceding and following pulse wave waveforms in the predetermined time, a variation in length of interval between the preceding and following pulse wave waveforms in the predetermined time, and a variation in difference in length of the interval between the preceding and following pulse wave waveforms in the predetermined time.

With such a configuration, it is possible to determine whether the arrhythmia occurring in the subject is the atrial fibrillation.

(4) The physiological information processing apparatus according to (2), in which after the arrhythmia of the subject is detected, the detector is configured to determine that premature contraction has occurred in the subject, in a case where a state where a difference between a height of a pulse wave waveform in a state where no arrhythmia occurs and a height of a pulse wave waveform based on newly acquired pulse wave data is less than a first threshold value continues for a predetermined time or more, or in a case where a state where which a difference between a length of an interval between pulse wave waveforms in a state where no arrhythmia occurs and a length of an interval between pulse wave waveforms based on the newly acquired pulse wave data is less than a second threshold value continues for a predetermined time or more.

With such a configuration, it is possible to determine whether the arrhythmia occurring in the subject is the premature contraction.

(5) The physiological information processing apparatus according to (2), in which after the arrhythmia of the subject is detected, the detector is configured to determine that tachycardia has occurred in the subject, in a case where a state where a height of a pulse wave waveform based on newly acquired pulse wave data is less than a third threshold value continues for a predetermined time or more, or in a case where a state where an interval between pulse wave waveforms based on the newly acquired pulse wave data is less than a fourth threshold value continues for a predetermined time or more.

With such a configuration, it is possible to determine whether the arrhythmia occurring in the subject is the tachycardia.

(6) A physiological information processing method executed by a physiological information processing apparatus, the physiological information processing method including:

acquiring pulse wave data indicating a pulse wave waveform of a subject; and

comparing a first pulse wave waveform with a second pulse wave waveform next to the first pulse wave waveform based on the pulse wave data to detect an arrhythmia of the subject.

With such a method, it is possible to detect the arrhythmia of the subject using the pulse wave data of the subject without acquiring electrocardiogram data.

(7) A non-transitory computer readable storage medium storing a computer program including at least one command executed by a processor of a physiological information processing apparatus, the computer program causing the physiological information processing apparatus to:

acquire pulse wave data indicating a pulse wave waveform of a subject; and

compare a first pulse wave waveform with a second pulse wave waveform next to the first pulse wave waveform based on the pulse wave data to detect an arrhythmia of the subject.

With such a configuration, it is possible to detect the arrhythmia of the subject using the pulse wave data of the subject without acquiring electrocardiogram data.

What is claimed is:

1. A physiological information processing apparatus comprising:
an acquisition unit configured to acquire pulse wave data indicating a pulse wave waveform of a subject; and
a detector configured to compare a first pulse wave waveform with a second pulse wave waveform next to the first pulse wave waveform, based on the pulse wave data, to detect an arrhythmia of the subject.

2. The physiological information processing apparatus according to claim 1, wherein
the detector is configured to detect the arrhythmia of the subject, based on at least one of a height of the pulse wave waveform and a length of an interval between the first pulse wave waveform and the second pulse wave waveform.

3. The physiological information processing apparatus according to claim 2,
wherein after the arrhythmia of the subject is detected, the detector is configured to determine whether atrial fibrillation has occurred in the subject, based on at least one of a variation in height of each pulse wave waveform in a predetermined time, a variation in difference in height between preceding and following pulse wave waveforms in the predetermined time, a variation in length of interval between the preceding and following pulse wave waveforms in the predetermined time, and a variation in difference in length of the interval between the preceding and following pulse wave waveforms in the predetermined time.

4. The physiological information processing apparatus according to claim 2,
wherein after the arrhythmia of the subject is detected, the detector is configured to determine that premature contraction has occurred in the subject, in a case where a state where a difference between a height of a pulse wave waveform in a state where no arrhythmia occurs and a height of a pulse wave waveform based on newly acquired pulse wave data is less than a first threshold value continues for a predetermined time or more, or in a case where a state where which a difference between a length of an interval between pulse wave waveforms in a state where no arrhythmia occurs and a length of an interval between pulse wave waveforms based on the newly acquired pulse wave data is less than a second threshold value continues for a predetermined time or more.

5. The physiological information processing apparatus according to claim 2,
wherein after the arrhythmia of the subject is detected, the detector is configured to determine that tachycardia has occurred in the subject, in a case where a state where a height of a pulse wave waveform based on newly acquired pulse wave data is less than a third threshold value continues for a predetermined time or more, or in a case where a state where an interval between pulse wave waveforms based on the newly acquired pulse wave data is less than a fourth threshold value continues for a predetermined time or more.

6. A physiological information processing method executed by a physiological information processing apparatus, the physiological information processing method comprising:
acquiring pulse wave data indicating a pulse wave waveform of a subject; and
comparing a first pulse wave waveform with a second pulse wave waveform next to the first pulse wave waveform based on the pulse wave data to detect an arrhythmia of the subject.

7. A non-transitory computer readable storage medium storing a computer program including at least one command executed by a processor of a physiological information processing apparatus, the computer program causing the physiological information processing apparatus to:
acquire pulse wave data indicating a pulse wave waveform of a subject; and
compare a first pulse wave waveform with a second pulse wave waveform next to the first pulse wave waveform based on the pulse wave data to detect an arrhythmia of the subject.
